(19) United States
(12) Patent Application Publication
(10) Pub. No.: US 2008/0300428 A1
(43) Pub. Date: Dec. 4, 2008

(54) USE OF alpha,beta -UNSATURATED CARBONYL COMPOUNDS AS QUENCH REAGENTS FOR THE BIRCH REDUCTION

(76) Inventors: Ingo ORTMANN, Werne (DE); Claus Christian Haeselhoff, Gelsenkirchen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/130,166

(22) Filed: May 30, 2008

Related U.S. Application Data
(60) Provisional application No. 60/941,022, filed on May 31, 2007.

(51) Int. Cl. C07C 49/543; C07C 49/20

(52) U.S. Cl. 568/376; 568/417

(57) ABSTRACT

Use of α,β-unsaturated carbonyl compounds as quench reagents for the Birch reduction.

This application claims the benefit of the filing date of U.S. Provisional Application Ser. No. 60/941,022 filed May 31, 2007.

The present invention relates to the use of α,β-unsaturated carbonyl compounds, especially of mesityl oxide, as a quench reagent for the Birch reduction and to processes for quenching a Birch reaction.

The Birch reaction, the partial reduction of styryl double bonds, α,β-unsaturated carbonyl compounds and aromatic systems with alkali metals (lithium, sodium, potassium), is a common method in organic synthesis [Rabideau, Tetrahedron 1989, 45, 1579-1603].

The Birch reduction in liquid ammonia with addition of alcohols with different pKₐ values as proton donors has been found to be particularly useful.

Under these conditions, the stereoselective reduction of the double bond in steroids (styryl double bond and α,β-unsaturated carbonyls) is possible.

The Birch reduction therefore finds use especially in the synthesis of pharmacologically relevant steroids, for example mesterolone [17β-hydroxy-1α-methylandrostan-3-one], 3-methoxy-18-methyl-1,3,5(10)-estratrien-17β-ol, 3-methoxy-1,3,5(10)-estratrien-17α-ol, [D. Bryce-Smith et al. J. Chem. Soc. C, 1966, 154; A. R. Utke et al. J. Org. Chem. 1964, 29, 1261; P. Markov et al. Compt. Rend. 1967, 264 C, 1605; K. Junghans, Chem. Ber. 1976, 109, 395; A. L. Wilds et al. J. Amer. Chem. Soc. 1953, 75, 5366; H. L. Dryden et al., J. Org. Chem. 1961, 26, 3237; U.S. Pat. No. 3,959,322; U.S. Pat. No. 3,519,714; Rabideau et al., Org. Reactions 1992, 42, 1].

The “quenching”—which is understood to mean the ending of a reaction by the controlled destruction of one of the reactants—is achieved in the Birch reduction by the addition of alcohols, acetone, methyl crotonate [Subba Rao, G. S. R. & Ramanathan, H.; Indian J. Chem., Sect. B; 20B, 1089 (1981)], methyl iodide [G. Stork et al., J. Am. Chem. Soc. 1965, 87, 275; B. Basu et al., Tetrahedron Letters 1984, 25, 1195], among other reagents. Equally suitable for this step are also inorganic salts, for example ammonium chloride.

What is common to the prior art quenching methods is that hydrogen is released. Furthermore, the abovementioned protic quench reagents have a high reactivity, owing to which there can be considerable exothermicity in the quenching of the Birch reaction.

Specifically on the industrial scale, the hydrogen release and the exotherms which occur in the course of quenching of the Birch reaction can lead to a considerable safety risk (runaway, explosions).

In the light of the prior art, the objective technical problem to be solved by the present invention is that of providing a safe process for quenching the Birch reduction on the industrial scale.

According to the present invention, the object is achieved by the use of α,β-unsaturated carbonyl compounds, especially of mesityl oxide (4-methyl-3-penten-2-one), 2-cyclohexan-1-one and methyl vinyl ketone, as quench reagents for the Birch reduction. The present invention therefore also provides a process for quenching a Birch reaction. The quench reagents according to the invention feature a sufficiently moderate reactivity toward the active alkali metal species to be quenched in the Birch reduction and enable, through the low exothermicity, a safe reaction.

In the present invention, preference is given to using mesityl oxide (4-methyl-3-penten-2-one), 2-cyclohexen-1-one or methyl vinyl ketone as the quench reagent for the Birch reduction.

Moreover, the use of the quench reagents according to the present invention has the advantage that the formation of undesired by-products in the Birch reaction is suppressed or prevented. This is because mesityl oxide reacts exclusively with the alkali metal species to be quenched without either reacting with the reactant used in the Birch reduction (the starting material to be reduced) or with the product. Furthermore, no hydrogen, which can lead under some circumstances to side reactions, is released.

During the quenching operation of the Birch reaction, mesityl oxide forms neither reactive radicals (as is the case for the acetone) nor reactive alkoxide ions. There are thus no polymerization products or formation of other undesired by-products. In the quenching operation, mesityl oxide forms only the readily removable methyl isobutyl ketone, which is typically used as a solvent.

The mesityl oxide quench reagent which is preferred in the present invention is easy to use from a process technology point of view because it is infinitely miscible with the solvents used in the reaction (e.g. tetrahydrofuran) and in this respect can be added in liquid form with good meterability.

The use of α,β-unsaturated ketones, especially of mesityl oxide, as a quench reagent for the Birch reduction thus enables not only a safe quenching process employable on the industrial scale but also leads to qualitatively and quantitatively better results with regard to the product.

The examples which follow shall now specifically illustrate the advantages which are of particular significance in the context of the present invention.

### EXAMPLE 1

### Preparation of mesterolone (17β-hydroxy-1α-methylandrostan-3-one)

In a mixture of 50 ml of THF, 19.0 ml of t-butanol and 40 ml of ammonia, 2.01 g (289.58 mmol) of lithium are dissolved at −45° C. The mixture is admixed at −50° C. with a solution of 32 g (105.79 mmol) of 1α-methyl testosterone in 210 ml of THF. After the reaction has ended, it is quenched by adding 10 ml of mesityl oxide. The reaction mixture is warmed to +20° C. and added to a suspension of 16 g of ammonium chloride and 50 ml of water. After phase separation, the organic phase is introduced into 2 l of ice-water. The resulting suspension is adjusted to pH=approx. 3 with HCl. Thereafter, the precipitated solid is isolated, washed to neutrality with water and dried at 50° C.

Yield: 98-100% of weight of starting material.

### EXAMPLE 2

### Preparation of 3-methoxy-18-methyl-1,3,5(10)-estratrien-17β-ol

In a mixture of 660 ml of THF, 131 ml of aniline and 1113 ml (753 g) of ammonia, 16.57 g of lithium are dissolved at −48° C. The mixture is admixed at −35° C. with a solution of 285 g of 3-methoxy-18-methyl-1,3,5(10),8-estratetraen-17β-ol and 487 ml of THF. After the reaction has ended, it is quenched by adding 57 ml of mesityl oxide in 57 ml of THF. The reaction mixture is warmed to +20° C. and added to a suspension of 127.5 g of ammonium chloride and 356 ml of water. After phase separation and extracting the aqueous phases twice with 125 ml of THF each time, the combined organic phases are concentrated by half at approx. 200 mbar. Addition of n-butanol and further vacuum distillation remove THF completely. An end volume of approx. 1450 ml is established. The mixture is admixed with HCl until pH=approx. 3 is attained and then kept at 90° C. for 15 min. The solution is subsequently cooled to 0° C. The crystals (seed if appropriate) are isolated, washed with approx. 400 ml of ice-cold n-butanol and approx. 1 l of water and dried at 50° C.

Yield: 246 g

### EXAMPLE 3

### Preparation of 3-methoxy-1,3,5(10)-estratrien-17α-ol

In a mixture of 24 l of aniline and 186 kg of ammonia, 5.1 kg of calcium are dissolved at −40° C. The mixture is admixed at −35° C. with a solution of 24 kg of 3-methoxy-1,3,5(10),8-estratetraen-17α-ol in 232 l of THF. After the reaction has ended, it is quenched by adding 8 l of mesityl oxide in 8 l of THF. The reaction mixture is warmed to +20° C. and added to a mixture, cooled to +5° C., of 80 l of acetic acid, 32 l of conc. hydrochloric acid and 1730 l of water. The precipitated solid is isolated, washed to neutrality with water and dried at 30° C.

Yield: 23.4 kg

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of U.S. Provisional Application Ser. No. 60/941,022, filed May 31, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Use of an α,β-unsaturated carbonyl compound as a quench reagent for the Birch reduction.

2. Use according to claim 1, characterized in that the quench reagent is mesityl oxide, 2-cyclohexen-1-one or methyl vinyl ketone.

3. Use according to claim 1, characterized in that a steroid is reduced in the Birch reaction.

4. Process for quenching a Birch reaction, characterized in that the reaction is ended by adding to it an α,β-unsaturated carbonyl compound.

5. Process according to claim 4, characterized in that the α,β-unsaturated carbonyl compound is mesityl oxide, 2-cyclohexen-1-one or methyl vinyl ketone.
